(19) United States
(12) Patent Application Publication
(10) Pub. No.: US 2022/0378360 A1
(43) Pub. Date: Dec. 1, 2022

(54) COMBINED ULTRASONIC STIMULATION AND PHOTOMETRY DEVICE

(71) Applicant: The Board of Trustees of the Leland Stanford Junior University, Stanford, CA (US)

(72) Inventors: George Quintin STEDMAN, Mountain View, CA (US); Kamyar FIROUZI, Palo Alto, CA (US); Butrus T. KHURI-YAKUB, Palo Alto, CA (US); Luis DE LECEA, Stanford, CA (US); Keith Richard MURPHY, Pacifica, CA (US)

(21) Appl. No.: 17/755,138

(22) PCT Filed: Nov. 12, 2020
(86) PCT No.: PCT/US2020/060244

§ 371 Date: Apr. 21, 2022

Related U.S. Application Data
(60) Provisional application No. 62/936,195, filed on Nov. 15, 2019.

(51) Int. Cl. A61B 5/00; G10K 11/24

(52) U.S. Cl. CPC A61B 5/4064; A61B 5/0071; G10K 11/24

(57) ABSTRACT

Devices and methods are disclosed related to combined ultrasonic stimulation and photometry. A device can include an ultrasonic transducer and an optical delivery element integrated with the ultrasonic transducer. The ultrasonic transducer can deliver ultrasound energy at a tip of the optical delivery element. In certain embodiments, the optical delivery element can be an optical fiber. A detector can generate an indication of a cell response associated with the delivered ultrasound energy.

### CROSS REFERENCE TO PRIORITY APPLICATION

This application claims the benefit of priority of U.S. Provisional Patent Application No. 62/936,195, filed Nov. 15, 2019 and titled “COMBINED ULTRASONIC STIMULATION AND FIBER PHOTOMETRY DEVICE,” the disclosure of which is hereby incorporated by reference in its entirely.

### FEDERAL SUPPORT STATEMENT

This invention was made with Government support under contract MH11647001 awarded by the National Institutes of Health. The Government has certain rights in the invention.

### BACKGROUND

### Technological Field

The disclosed technology relates to ultrasound and photometry including related devices, systems, and methods.

### Description of Related Technology

Using ultrasound to non-invasively modulate behavior of a brain is a growing area of research. Studies have demonstrated that ultrasound can alter neuronal and brain-network activity. Neuromodulation using ultrasound has great potential to non-invasively treat a wide array of neurological disorders, including epilepsy, essential tremor, depression, and sleep disorders. However, detecting neuronal activity and/or an impact of ultrasound on specific brain cells has presented technical challenges. Accordingly, improved devices, systems, and methods for detecting responses to ultrasound delivered to brain cells would be desirable.

### SUMMARY OF CERTAIN INVENTIVE ASPECTS

The innovations described in the claims each have several aspects, no single one of which is solely responsible for its desirable attributes. Without limiting the scope of the claims, some prominent features of this disclosure will now be briefly described.

One aspect of the disclosed technology is a device for combined. ultrasound stimulation and photometry. The device includes an ultrasonic transducer, an optical delivery element integrated with the ultrasonic transducer, a light source configured to transmit light via the optical delivery element, and a detector coupled to the optical delivery element. The ultrasonic transducer is configured to deliver ultrasound energy at a tip of the optical delivery element. The detector is configured to generate an indication of a cell response associated with the delivered ultrasound energy.

The optical delivery element can include an optical fiber. The ultrasonic transducer can be configured to deliver ultrasound energy via the optical element. The optical delivery element can be brain implantable. The ultrasonic transducer can include a gradient index lens.

The light source can include an excitation laser. The detector can include one or more photodetectors. The device can include a cannula arranged to hold the optical fiber. The device can include a sleeve arranged to hold the optical delivery element and a ferrule connected to the cannula. The ultrasonic transducer can include a piezoelectric element positioned between the ferrule and the cannula. The piezoelectric element can includes an opening arranged to allow light to pass between the ferrule and cannula.

Another aspect of the disclosed technology is a device for combined ultrasound stimulation and photometry. The device includes an ultrasonic transducer and an optical delivery element integrated with the ultrasonic transducer. The ultrasonic transducer is configured to deliver ultrasound energy at a tip of the optical delivery element. The optical delivery element is configured to carry light from a light source.

The optical delivery element can include an optical fiber. The optical delivery element can be brain implantable. The ultrasonic transducer can be configured to deliver ultrasound energy via the optical delivery clement. The device can include a cannula arranged to hold the optical delivery element. The ultrasonic transducer can include a piezoelectric element including an opening arranged to allow light to pass through.

Another aspect of the disclosed technology is a method of combined ultrasound stimulation and photometry. The method includes applying ultrasound energy to a region of a brain at a tip of an optical delivery element, transmitting light from a light source to the region of the brain using the optical delivery element, and detecting, with a detector, a response to the applied ultrasound energy.

The method can include determining one or more ultrasonic stimulation parameters that excite a particular cell type based on the detecting. The method can include determining one or more ultrasonic stimulation parameters that inhibit a particular cell type based on the detecting.

The applying ultrasound energy can be performed via the optical delivery element. The optical delivery element can include an optical fiber. The optical fiber can be integrated with an ultrasonic transducer. The optical fiber can be implanted in the brain. The ultrasonic transducer can deliver ultrasound through the optical fiber, providing ultrasonic stimulation in a relatively small region at the end of the tip of the optical fiber. At the same time, an excitation laser and calcium dependent fluorescence emissions from neuronal sub-types can be transmitted through the optical fiber. This can allows for the measurement to be of cell-type specific response to ultrasonic stimulation in spatially controlled regions of the brain.

Another aspect of the disclosed technology is a method of combined ultrasound stimulation and measurement. The method includes applying ultrasound energy to a region of a brain using an ultrasonic transducer and measuring a response to the applied ultrasound energy in the region of the brain with single cell resolution.

The measuring can include using a gradient-index lens. The gradient-index lens can be embedded in a cannula that is in electrical communication with an electrode of the ultrasonic transducer. The measuring can include using photoacoustics. The measuring can include using a fiber photometry device integrated with the ultrasonic transducer.

For purposes of summarizing the disclosure, certain aspects, advantages and novel features of the innovations have been described herein. It is to be understood that not necessarily all such advantages may be achieved in accordance with any particular embodiment. Thus, the innovations may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1A illustrates a combined ultrasonic stimulation and fiber photometry device according to an embodiment

FIG. 1B is a diagram illustrating cell-type specific labeling of cells exposed to ultrasound simulation an optical GCaMP excitation light.

FIG. 1C illustrates a computational model of acoustic pressure at the fiber optic tip of the optical fiber of the combined ultrasonic stimulation and fiber photometry device of FIG. 1A.

FIG. 2A illustrates a combined ultrasonic stimulation and fiber photometry device according to an embodiment.

FIG. 2B shows an acoustic intensity around a fiber tip of the combined ultrasonic stimulation and fiber photometry device of FIG. 2A.

FIG. 3A illustrates an exploded view of the combined ultrasound and photometry device.

FIG. 3B illustrates an assembled view of the combined ultrasound and photometry device of FIG. 3A.

FIG. 4A provides measurement results of a hydrophone measurement of ultrasound applied using the device of FIGS. 3A and 3B.

FIG. 4B is a graph of temperature modeling showing change in temperature range over time for applied ultrasound with the device of FIGS. 3A and 3B.

FIG. 5A includes graphs measuring GCaMP7 fluorescence changes in calcium/calmodulin-dependent protein kinase II (CamKII) positive cells of the central medial thalamus during ultrasound of varying pulse length.

FIG. 5B includes graphs measuring GCaMP7 fluorescence changes in CamKII positive cells of the central medial thalamus during ultrasound of varying maximum pulse peak-to-peak pressure.

FIGS. 6A and 6B compare ultrasound response in CAMKII positive cells of a central medial thalamus to CAMKII positive cells of a CA1 in a hippocampus.

FIG. 7A is a graph of probability of sleep over time for ultrasound stimulus and a control.

FIG. 7B is a graph of state specific electroencephalogram power spectra recorded during a baseline period compared with an ultrasound stimulation period.

### DETAILED DESCRIPTION OF CERTAIN EMBODIMENTS

The following detailed description of certain embodiments presents various descriptions of specific embodiments. However, the innovations described herein can be embodied in a multitude of different ways, for example, as defined and covered by the claims. In this description, reference is made to the drawings where like reference numerals and/or terms can indicate identical or functionally similar elements. It will be understood that elements illustrated in the figures are not necessarily drawn to scale. Moreover, it will be understood that certain embodiments can include more elements than illustrated in a drawing and/or a subset of the elements illustrated in a drawing. Further, some embodiments can incorporate any suitable combination of features from two or more drawings.

The use of ultrasound to non-invasively modulate behavior of the brain is a growing area of research given its high spatial and temporal specificity. Many studies have demonstrated that select intensities, frequencies, and modulation patterns of ultrasound can alter neuronal and brain-network activity. Neuromodulation using ultrasound therefore has great potential to non-invasively treat a wide array of neurological disorders, including epilepsy, essential tremor, depression, and sleep disorders.

The select intensities, frequencies, and modulation patterns that can enhance and/or suppress neurological activity are an open area of study and have not yet been well-established. Furthermore, how functionally distinct cell-types respond to these ultrasound waveform features is almost entirely unknown. This shortcoming is a barrier to ultrasound neuromodulation replacing and/or supplementing pharmacological psychiatric treatments.

Electrical recording from populations of neurons can lack the capacity to distinguish the genetically defined cell-types generating the recorded signal. In this case, the signaled neural response to a given treatment may not be emanating from a desired target. Optical readouts of neuronal activity have become a significant tool in neuroscience, largely because they can be genetically targeted to specific cell-types. However, optical access to sub-superficial regions of the brain is limited due to the light-scattering effects of tissue. To circumvent this limitation, implanting optical fiber into the brain has become a popular approach for optically measuring sub-superficial cell type activity. This method also benefits from its ability to transmit the signal over a distance using a light-weight implant and fiber optic cable, allowing the animals to behave freely during recording. However, this method poses a significant issue for the delivery of ultrasonic waves as the fiber can block and/or diffract waves passing from tissue across the fiber as they penetrate the brain. Thus, accurately measuring sub-superficial cell-type response to ultrasound is currently infeasible.

Combining ultrasonic stimulation with fiber photometry in the same device is disclosed herein. A device capable of delivering ultrasonic neuromodulation to highly restricted areas of the brain while simultaneously recording neuronal activity with cell-type specificity is disclosed. The combined ultrasonic stimulation and fiber photometry devices disclosed herein can be referred to as combined ultrasonic stimulation with fiber photometry probes.

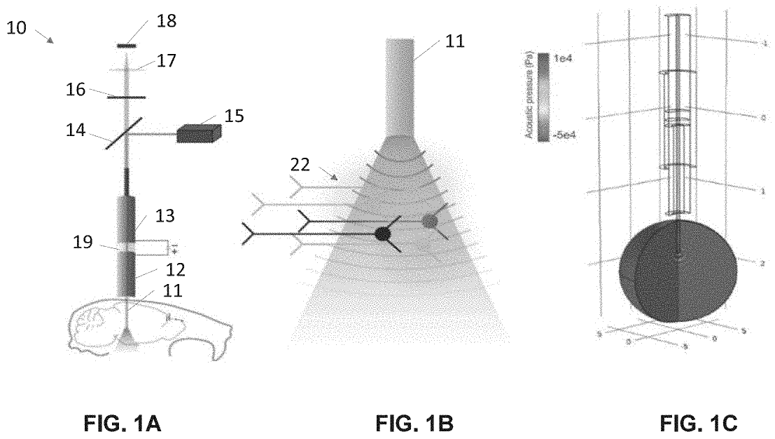

A device for combined ultrasonic stimulation and fiber photometry can be a modification of a fiber photometry system. FIG. 1A illustrates a combined ultrasonic stimulation and fiber photometry device 10 according to an embodiment.

A combined ultrasonic stimulation and fiber photometry device 10 is shown schematically in FIG. 1A. An optical fiber 1l can be implanted in the brain of the subject, such as a mouse. Accordingly, the optical fiber 11 is an example of a brain implantable optical delivery element. In the combined ultrasonic stimulation and fiber photometry device 10, the optical fiber 11 is held by a cannula 12. The cannula 12 can be a steel cannula. A ferrule 13 is connected to the cannula 12 using a sleeve that slides over the ferrule 13 and the cannula 12. Accordingly, the ferrule 13 may be easily attached to and/or detached from the cannula 12. The ferrule 13 can be a steel ferrule. The fibers in the cannula 12 and ferrule 13 are aligned so that light can pass between them, connecting the fiber tip with the photometry system.

A fiber photometry system includes the optical fiber 11, the cannula 12, and the ferrule 13 connected to the cannula 12 by a sleeve, a dichroic mirror 14, a light source 15, a bandpass filter 16, a lens 17, and a photodetector 18. The light source 15 can provide light to the brain of the subject via the optical fiber 11. The light source 15 can be a laser, such as a 473 nanometer laser. The light source 15 can be any other suitable light source. The dichroic mirror 14 can direct light from the light source 15 toward the brain. The dichroic mirror can direct light from the brain to the photodetector 18 via the bandpass filter 16 and the lens 17. The photodetector 18 can detect a cell response to ultrasound applied to the brain. In certain instances, the photodetector 18 can detect a cell response with single cell resolution.

According to an embodiment, an ultrasonic transducer is integrated into the optical probe. For example, as shown in FIG. 1A, a ring-shaped piezoelectric element 19 is included between the cannula 12 and ferrule 13. The ferrule 13 and cannula 12 can be metallic and provide electrical connections to the electrodes on the top and bottom of the piezoelectric element 19. The open center of the ring of the piezoelectric element 19 can allow light to pass between the ferrule 13 and cannula 12. When the piezoelectric element 19 is excited with a voltage, the piezoelectric element 19 sends ultrasonic energy into the cannula 12 and the optical fiber 11. This can create an acoustic field in a volume at the tip of the optical fiber 11. The piezoelectric element 19 can be sufficiently thin such that the piezoelectric element 19 does not significantly compromise the photometry signal.

FIG. 1B is a diagram illustrating cell-type specific labeling of cells exposed to ultrasound simulation an optical GCaMP excitation light. The labeled cells 22 are exposed to ultrasound simulation and GCaMP excitation light near a tip of the optical fiber 11 of FIG. 1A. GCaMP is a genetically coded calcium indicator.

FIG. 1C illustrates a computational model of acoustic pressure at the fiber optic tip of the optical fiber 11 of FIG. 1A. The computation model of FIG. 1C demonstrates highly localized pressure.

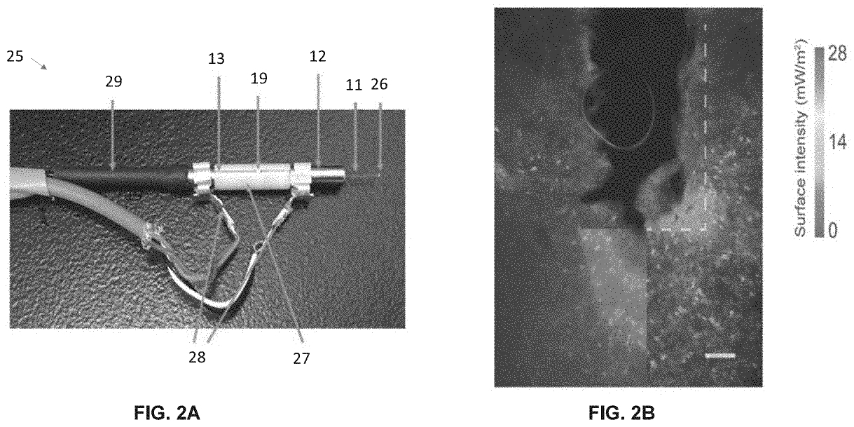

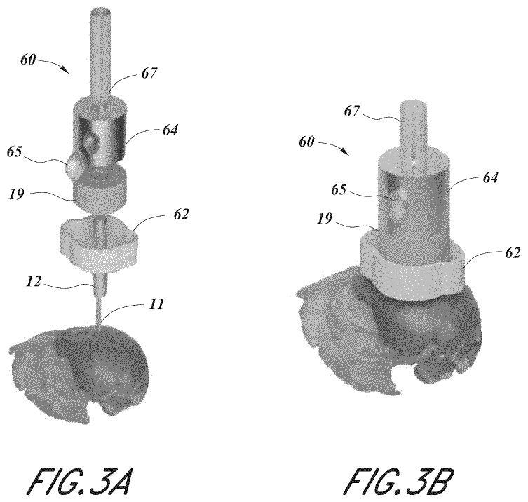

FIG. 2A shows an embodiment of a combined ultrasonic stimulation and fiber photometry device 25. As illustrated, the combined ultrasonic stimulation and fiber photometry device 25 includes an optical fiber 11 with a tip 26, a cannula 12, a ferrule 13 connected to the cannula 12 using a sleeve 27, an ultrasonic transducer, electrical leads 28 providing electrical connections to the cannula 12 and ferrule 13, and a fiber patch cord 29. In the combined ultrasonic stimulation and fiber photometry device 25, the ultrasonic transducer is a piezoelectric element 19. Any other suitable ultrasonic transducer can be used in place of the piezoelectric element 19 in certain applications. The ultrasonic transducer can deliver ultrasound energy to a region of a brain and the fiber photometry system of the device 25 can detect a cell response to the delivered ultrasound energy. The illustrated device 25 is not connected to a subject.

The device 25 of FIG. 2A was modeled using the finite element method. FIG. 2B shows the acoustic intensity around the fiber tip of the device 25 as the tip moves at 2.4 MHz with a displacement of 1 nm in the normal direction overlaid on a brain image of labeled cells and a fiber tract. This shows that the region of high acoustic intensity is confined to the region around the fiber tip where genetically labeled cells are localized. FIG. 2B illustrates an image of Cre-recombinase dependent viral labeling of hypocretin cells with an overlay of acoustic field intensity modeled from LDV surface pressure measurements. In FIG. 2B, a half pressure field is shown. The tear in the brain is remnant of the placement of the optical fiber 11. The scale bar is 100 micrometer.

The ability of the device to produce an ultrasonic excitation at the fiber tip has been demonstrated by measuring the tip displacement with a laser Doppler vibrometer while the piezoelectric element is excited.

A combined ultrasonic stimulation and fiber photometry device has been tested on an awake mouse with a genetically encoded fluorescent calcium indicator in the cells immediately below the fiber tip. The mouse moved freely with the device attached, the photometry measurement was not impeded, and the mouse showed no ill effects when an excitation voltage was applied across the piezoelectric element. A neuronal response to ultrasonic delivery can be measured while delivering ultrasound with various parameters including, but not limited to, carrier frequencies, frequency or amplitude modulation frequencies, pressures or intensities.

A combined ultrasonic stimulation and fiber photometry according to embodiments enables measurement of the response of defined cell types in a particular location of the brain. Delivering the ultrasound through an optical fiber ensures alignment of the ultrasonic stimulation with the optical measurement area and also ensures that the ultrasound is spatially confined. The skull makes it difficult to focus ultrasound to a precisely located, spatially confined volume using a separate external transducer. Even without the skull, it would be difficult to align the field precisely to the optical fiber tip, and the tip could itself reflect ultrasound, further complicating the problem.

Combined ultrasonic stimulation and fiber photometry devices disclosed herein can be lightweight. Accordingly, such devices can be used on awake, freely moving animals. This is significant for observing the behavioral effects of ultrasonic stimulation and can avoid the confounding effect of anesthesia and/or physical constraint.

A device in accordance with the principles and advantages disclosed herein can be used to identify ultrasonic stimulation parameters that excite and/or inhibit particular cell types. Accordingly, precisely targeted treatments for neurological disorders can be developed by targeting particular cell types. Cell-specific ultrasonic neuromodulation parameters can be identified to treat a variety of conditions, such as one or more of sleep disorders, epilepsy, essential tremor, Alzheimer's, depression, or general psychiatric disorders.

Other suitable methods of ultrasonic transduction could be used in combined ultrasonic stimulation and fiber photometry devices. For example, ultrasonic transduction based on optical absorption could be used. In such an instantiation, a light absorbent ring, whose inner diameter partially overlaps with the optical fiber's light path, could be placed between the cannula and ferrule in place of a piezoelectric element. The material could then be excited with a pulsed laser, producing ultrasound. Alternatively, the material could be selected so that it is transparent to the wavelengths used for photometry but opaque to the wavelength of the pulsed laser so that the material does not compromise the quality of the photometry. In another instantiation, a photo-absorbent material could be placed at the tip of the fiber to achieve a similar effect.

In an embodiment, the ferrule, piezoelectric element, and cannula are held in place with a sleeve so that the fiber can be easily attached and detached from the subject. In some cases, it may be advantageous to fix the piezoelectric element to the cannula and/or ferrule with an adhesive to improve the ultrasound coupling into the probe. Alternatively, it may be advantageous to include a device to push the ferrule and the cannula together. This could be a small clamp or one or more magnets, for example. A clamp may help secure the sleeve surrounding the cannula and ferrule to prevent the parts from separating during ultrasound delivery.

In another embodiment of the device, the optical fiber may be replaced with a Gradient-Index (GRIN) lens embedded in a cannula. This can allow for single cell resolution when coupled to a miniaturized microscope or with a head-fixed animal below an immobile microscope. An optical delivery element can include a GRIN lens and/or an optical fiber. According to another embodiment, the device can include photoacoustics in place of the optical fiber. The devices disclosed herein can be adapted to include any suitable hardware to measure a cell specific response to applied ultrasound.

Ultrasound can be applied at a tip of an optical delivery element (e.g, at a tip of an optical fiber or a tip of a GRIN lens). In certain embodiments, ultrasound can be applied via an optical delivery element, such as an optical fiber, resulting in ultrasound being applied at a tip of the optical delivery element. In some other embodiments, one or more ultrasonic transducers can apply ultrasound at a tip of the optical delivery element via a path separate from the optical delivery element. The tip of the optical delivery element can be used as a registration point for coordinating position of the ultrasonic transducer to tissue for applying ultrasound.

In an embodiment, an ultrasonic transducer is externally coupled or mounted directly or indirectly to a cannula housing of an implanted optical fiber with the ultrasound propagating compression waves in parallel to the cannula housing and fiber. Such ultrasound can have a larger focal spot and/or potentially greater intensity than ultrasound applied via an optical fiber. The ultrasound transducer can control the power, size, and/or shape of the ultrasound field applied at the tip of the optical filter. The ultrasonic transducer can be secured to a fiber optic cannula through friction, hardware, or a bonding agent. If the ultrasonic transducer is not directly coupled to a skull with a bonding agent, an acoustic coupling agent may be used to reduce acoustic reflection and/or reflection caused by an air interface between the skull and the ultrasonic transducer.

The fiber optic probe can be housed in within a cannula. The fiber optic probe can be embedded directly in the ultrasonic transducer. The fiber optic implant can serve as a registration point for coordinating the position of the ultrasonic transducer to the brain tissue below the fiber probe tip.

The ultrasonic transducer can be a cylindrical ring, rectangular, an amorphous shape, or any other suitable shape. The ultrasonic transducer can be a capacitive micromachined transducer, a single element piezoelectric transducer or piezoelectric stack, a piezoelectric array, one or inure piezoelectric micromachined ultrasonic transducers, or any other suitable ultrasonic transducer. The ultrasonic transducer can be fitted with a backing and/or matching layer to improve output pressure and/or modify an aspect of the ultrasound field.

The elements of an ultrasonic transducer array can be excited at different temporal phase to achieve beam steering and/or focusing. The ultrasonic transducer element(s) can form a natural focus at the tip of the fiber, such as the case for a single element ring ultrasonic transducer. The ultrasound field and/or focus can be modified by changing the dimensions of the ultrasonic transducer. The ultrasonic transducer can be driven such that a focused or unfocused beam profile reaches the tissue below the fiber optic tip such that the proximal cells provide a readout of cell response to the ultrasound activity.

The ultrasound field can be targeted to tissue not directly below the optical fiber in order to measure interactions between ultrasonic stimulation of external cells and the cells below the optical fiber tip. The ultrasonic field may affect a sufficient volume of tissue to affect behavior.

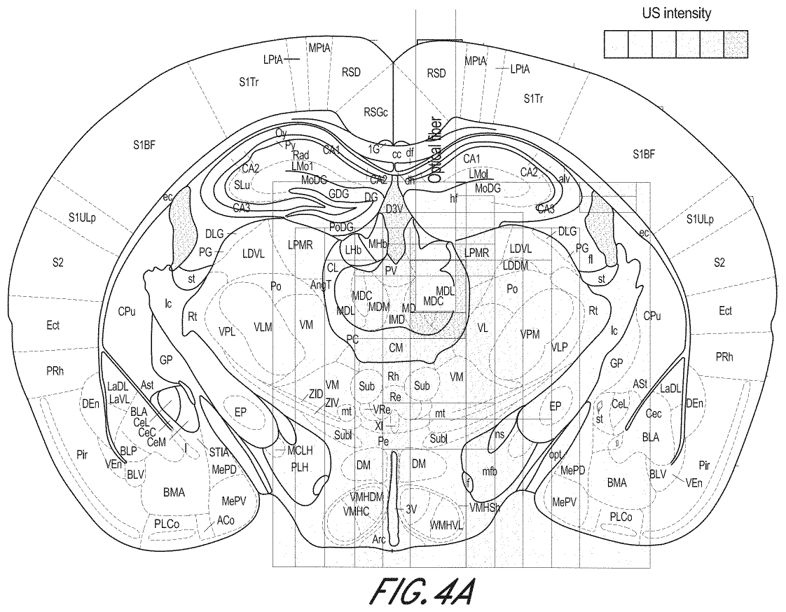

FIGS. 3A and 3B illustrate a combined ultrasound and photometry device 60 according to an embodiment. FIG. 3A illustrates an exploded view of the combined ultrasound and photometry device 60. FIG. 3B illustrates an assembled view of the combined ultrasound and photometry device 60.

The combined ultrasound and photometry device 60 includes an optical fiber 11, a cannula 12, a piezoelectric element 19, an acoustic coupling gel bracket 62, a mounting clamp 64, a clamp screw 65 and a cannula sleeve 67. The piezoelectric element 19 includes a transcranial piezoelectric ring in the device 60. The piezoelectric element 19 is mounted to the cannula 12 in the device 60. The piezoelectric element 19 can generate ultrasound waves propagating generally in parallel to housing of the cannula housing 12 and the optical fiber 11. The ultrasound can be delivered to tissue at a tip of the optical fiber 11. The clamp 64 can hold the piezoelectric element 19 in place. The clamp 64 is a collar clamp as illustrated.

The combined ultrasound and photometry device 60 can be surgically installed. For example, the optical fiber 11 can be implanted into a mouse brain. The cannula 12 and the optical fiber 11 can be stereotactically implanted above the central medial thalamus (CMT), in which case the optical fiber 11 projects into a brain just above the CMT. A virus encoding an optical neuronal activity indicator can be delivered to the CMT via a silk coating on the tip of the optical fiber 11. The clamp 64 is attached to the fiber optic cannula via the clamp screw 65. The ultrasonic transducer face of the piezoelectric element 19 can be acoustically coupled to a skull using dielectric grease. A ring focus of the piezoelectric element 19 can be centered above the focal point of the CMT. A height of the piezoelectric element 19 can be adjusted by sliding the clamp 64 up and/or down the cannula 12.

In certain applications, the optical fiber 11 can be used for calibration of ultrasound parameters for optimal neural stimulation. Then the optical fiber can be removed and ultrasound can be applied for neuromodulation without the optical fiber being implanted in the brain.

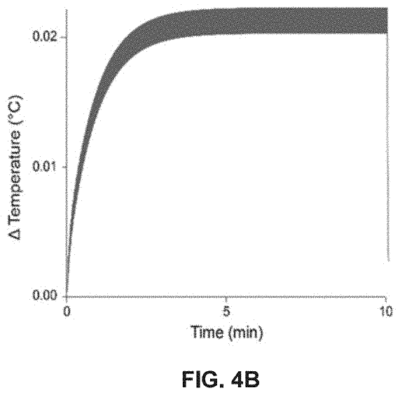

A focused ultrasound field was characterized using a hydrophone below a skull surface of an explanted skull mounted to the device. FIG. 4A provides measurement results of a hydrophone measurement of ultrasound ring focus overlaying a target region. The central medial thalamus is outlined with a dark line in FIG. 4A. The applied ultrasound field delivered a peak pressure of over 2 MPa for obtaining the results of FIG. 4A.

FIG. 4B is a graph of temperature modeling showing change in temperature range over time for applied ultrasound with the device 60. The thermal modeling indicates that the pressure field produces negligible (about <0.025° C.) changes in temperature at 10% duty cycle.

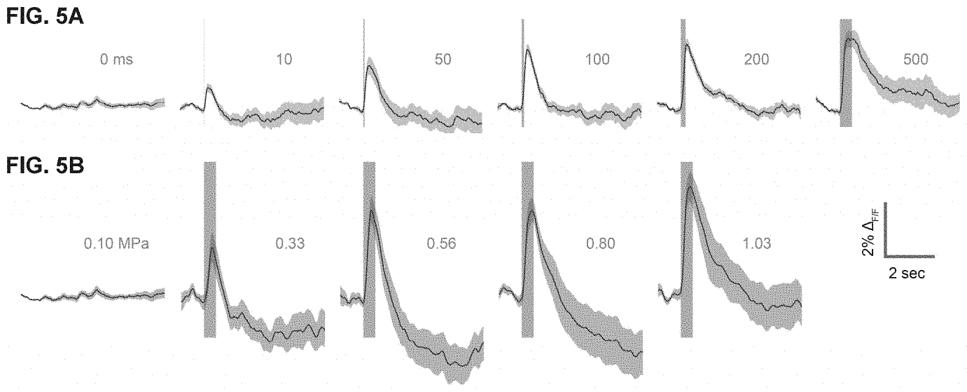

Using the combined ultrasound and photometry device 60, fiber photometry was used to perform calcium-based fluorescence measurements as a proxy of neural activity. FIG. 5A contains graphs measuring GCaMP7 fluorescence changes in calcium/calmodulin-dependent protein kinase 11 (CamKII) positive cells of the central medial thalamus during ultrasound of varying pulse length. FIG. 5B contains graphs measuring GCaMP7 fluorescence changes in CamKII positive cells of the central medial thalamus during ultrasound of varying maximum pulse peak-to-peak pressure. Each line represents the mean of 10 trials per duration and the shading represents the standard error of the mean. The trials were performed with a duty cycle of 100% and carrier frequency of 623 KHz.

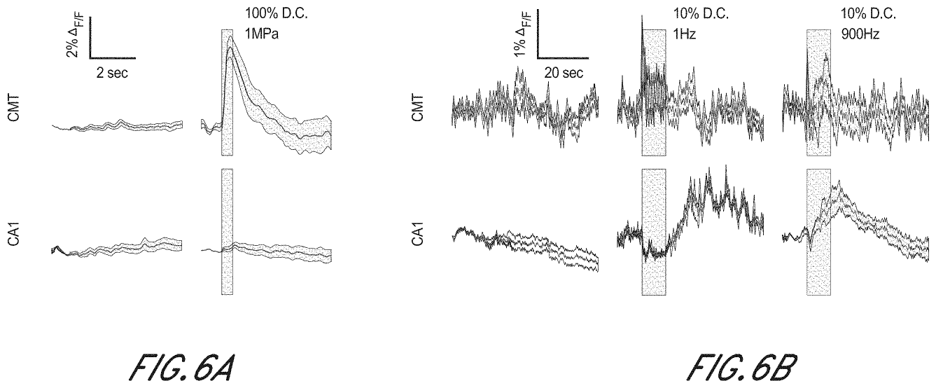

FIGS. 6A and 6B compare ultrasound response in CAMKII positive cells of the central medial thalamus to CAMKII positive cells of the CA1 in the hippocampus. FIG. 6A shows the response of the two cell types to 500 ms continuous pulse at ˜1 MPa peak-to-peak pressure. While the thalamic cells show a large response to the pulse, hippocampal cells show virtually none. FIG. 6B corresponds to a 10% duty cycle waveform with a pulse repetition frequency of 1 Hz or 900 Hz for 10 seconds. At 1 Hz, the thalamic cells show an immediate increase in activity with visible spikes following the 1 Hz pulse repetition frequency. In contrast, the hippocampal cells show a bimodal response with an immediate reduction in activity followed by an increase following the stimulation period. At 900 Hz, both cell types show an immediate increase in activity, with the hippocampal cells increase continuing after the stimulation period. These graphs indicate that various cell types had different responses to various ultrasonic waveforms which could be quantified by a device according to an embodiment.

By simultaneously recording sleep with an electroencephalogram (EEG) readout in the same animals, it was found that 10 minutes of pulsed ultrasound stimulation of the thalamus of a freely behaving mouse transiently increased the animal's probability of sleep or decreased the probability of wakefulness, measured by EEG analysis.

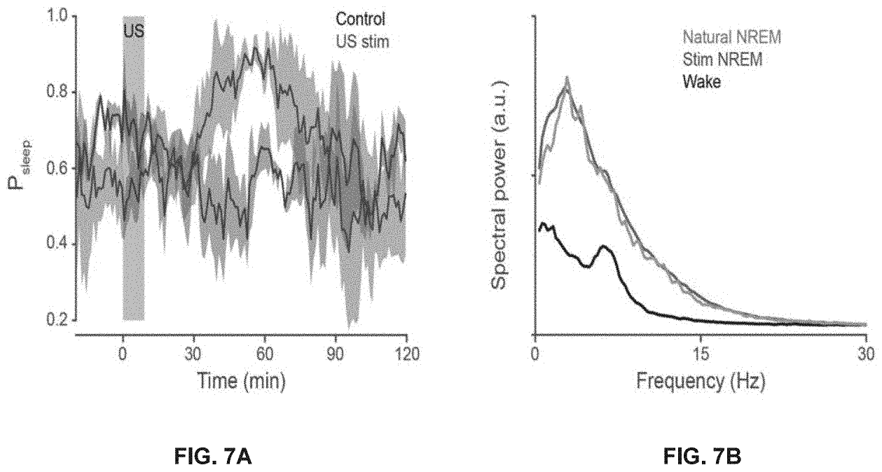

FIG. 7A is a graph of probability of sleep over time for ultrasound stimulus and a control. The ultrasound stimulus had an intensity, spatial-peak pulse-average (ISPPA) of 2.3 W/cm², 0.37 MPa peak pressure, a carrier frequency of 711 KHz, and a modulation frequency of 3 Hz with 10% duty cycle. There was n=3 with 7 trials per condition. FIG. 7A indicates that the ultrasound stimulus increased the probability of sleep.

FIG. 7B is a graph of state specific EEG power spectra recorded during a baseline period compared with an ultrasound stimulation period. The baseline period was 10 minutes prior to ultrasound stimulation. The ultrasound stimulation period was 0 to 90 minutes. FIG. 7B indicates that the stimulation induced sleep was characteristically indistinguishable from natural sleep in tennis of power spectral distribution.

Embodiments disclosed herein can provide data regarding consequences of ultrasound delivery to brain location(s). This can provide information about effects of focused ultrasound on specific brain location(s). Devices disclosed herein can serve as a clinical research tool for identifying the precise ultrasonic waveforms for modulation of one or more neuronal subtypes, or all subtypes, in defined regions of the brain. Identified waveforms can then be delivered to specific regions of the brain by a transcranial and/or subcranial ultrasonic transducer, and/or an implantable ultrasonic emitting device. This device can identify such waveforms using any animal subject in which fiber optic probes can be surgically implanted. One example of this usage would be identifying a waveform which preferentially inhibits hypocretin/orexin cells of the lateral hypothalamus. The waveforms can then be delivered in human patients using focused ultrasound to improve sleep quality through the cell-type specific inhibition. Using this device to improve waveform selection may enable and/or substantially improve use of ultrasonic neuromodulation for a broad array of psychiatric and/or other disorders.

While certain embodiments have been described, these embodiments have been presented by way of example only and are not intended to limit the scope of the disclosure. Indeed, the novel devices, systems, apparatus, and methods described herein may be embodied in a variety of other forms. The principles and advantages of the embodiments can be used for any other suitable devices, systems, apparatuses, and/or methods that could benefit from such principles and advantages. Furthermore, various omissions, substitutions and changes in the form of the methods and systems described herein may be made without departing from the spirit of the disclosure. All possible combinations and sub combinations are intended to fall within the scope of this disclosure. For example, while blocks are presented in a given arrangement, alternative embodiments may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further embodiments.

1. A device for combined ultrasound stimulation and photometry, the device comprising:
an ultrasonic transducer;
an optical delivery element integrated with the ultrasonic transducer, the ultrasonic transducer configured to deliver ultrasound energy at a tip of the optical delivery element;
a light source configured to transmit light via the optical delivery element; and
a detector coupled to the optical delivery element, the detector configured to generate an indication of a cell response associated with the delivered ultrasound energy.

2. The device of claim 1, wherein the optical delivery element comprises an optical fiber.

3. The device of claim 2, wherein the ultrasonic transducer is configured to deliver the ultrasound energy via the optical fiber.

4. The device of claim 1, wherein the optical delivery element is brain implantable.

5. The device of claim 1, wherein the ultrasonic transducer is configured to deliver ultrasound energy via the optical delivery element.

6. The device of claim 1, wherein the optical delivery element comprises a gradient-index lens.

7. The device of claim 1, wherein the light source comprises an excitation laser, and the detector comprises one or more photodetectors.

8. The device of claim 1, further comprising a cannula arranged to hold the optical delivery element.

9. The device of claim 8, further comprising a ferrule connected with the cannula, wherein the ultrasonic transducer comprises a piezoelectric element positioned between the ferrule and the cannula.

10. The device of claim 9, wherein the piezoelectric element comprises an opening arranged to allow light to pass between the ferrule and cannula.

11. (canceled)

12. (canceled)

13. (canceled)

14. (canceled)

15. (canceled)

16. (canceled)

17. A method of combined ultrasound stimulation and photometry, the method comprising:
applying ultrasound energy to a region of a brain at a tip of an optical delivery element;
transmitting light from a light source to the region of the brain using the optical delivery element; and
detecting, with a detector, a response to the applied ultrasound energy.

18. The method of claim 17, further comprising determining one or more ultrasonic stimulation parameters that excite a particular cell type based on the detecting.

19. The method of claim 17, further comprising determining one or more ultrasonic stimulation parameters that inhibit a particular cell type based on the detecting.

20. The method of claim 17, wherein the applying ultrasound energy is performed via the optical delivery element.

21. The method of claim 17, wherein the optical delivery element comprises an optical fiber.

22. A method of combined ultrasound stimulation and measurement, the method comprising:
applying ultrasound energy to a region of a brain using an ultrasonic transducer; and
measuring a response to the applied ultrasound energy in the region of the brain with single cell resolution.

23. The method of claim 22, wherein the measuring comprises using a gradient-index lens.

24. The method of claim 23, wherein the gradient-index lens is embedded in a cannula that is in electrical communication with an electrode of the ultrasonic transducer.

25. The method of claim 22, wherein the measuring comprises using photoacoustics.

26. The method of claim 22, wherein the measuring comprises using a fiber photometry device integrated with the ultrasonic transducer.
